(19) United States
(12) Patent Application Publication
(10) Pub. No.: US 2016/0340334 A1
(43) Pub. Date: Nov. 24, 2016

(54) METHOD FOR ISOLATION OF ALKALOIDS AND AMINO ACIDS, AND COMPOSITIONS CONTAINING ISOLATED ALKALOIDS AND AMINO ACIDS

(71) Applicant: Joseph Robert Knight, Las Vegas, NV (US)

(72) Inventor: Joseph Robert Knight, Las Vegas, NV (US)

(21) Appl. No.: 15/195,490

(22) Filed: Jun. 28, 2016

Related U.S. Application Data
(63) Continuation of application No. PCT/US2016/033039, filed on May 18, 2016.
(60) Provisional application No. 62/179,763, filed on May 19, 2015.
(60) Provisional application No. 62/179,764, filed on May 19, 2015.
(60) Provisional application No. 62/179,765, filed on May 19, 2015.
(60) Provisional application No. 62/179,766, filed on May 19, 2015.
(60) Provisional application No. 62/285,094, filed on Oct. 19, 2015.

(51) Int. Cl. C07D 401/04; C07D 311/80; A61M 15/06; A24B 15/16; A23L 2/52; C07D 209/20; A24F 47/00

(52) U.S. Cl. CPC C07D 401/04; C07D 209/20; C07D 311/80; A24F 47/008; A24B 15/16; A23L 2/52; A61M 15/06; A23V 2002/00

(57) ABSTRACT

Described herein are methods to purify alkaloids and amino acids. Also described are compositions and methods to treat or prevent a disease using the purified amino acids and alkaloids. The composition can be, for example, formulated into a beverage, a supplement, or formulated for use in e-cigarettes. Also described are methods of treating a disease such as Parkinson's Disease, Alzheimer's Disease, ADHD, ADD, vascular disorders, cancer, heart disorders, migraine headaches, cluster headaches, plaque reduction or aging using one or more of the purified alkaloids or amino acids.

### CROSS REFERENCE TO RELATED APPLICATIONS

This application is a continuation of International Patent Application No. PCT/US2016/033039 titled “Method for Isolation of Alkaloids and Amino Acids, and Compositions Containing Isolated Alkaloids and Amino Acids,” filed May 18, 2016, which claims the benefit of U.S. Provisional Patent Application No. 62/179,763 filed on May 19, 2015; U.S. Provisional Patent Application No. 62/179,764 filed on May 19, 2015; U.S. Provisional Patent Application No. 62/179,765 filed on May 19, 2015; U.S. Provisional Patent Application No. 62/179,766 filed on May 19, 2015; and U.S. Provisional Patent Application No. 62/285,094 filed on Oct. 19, 2015, the contents of which are incorporated herein by reference in their entirety.

### BACKGROUND

It is known that amino acids and alkaloids can be used to treat migraines and cluster headaches, as well as other disorders such as Parkinson's Disease, Alzheimer's Disease, attention deficit/hyperactivity disorder (ADHD), attention deficit disorder (ADD), vascular disorders, cancer, heart disorders, tissue detoxification, plaque reduction and aging.

However, to date, the use of amino acids and alkaloids such as nicotine, caffeine, cannabis, morphine, and cocaine have not been successful as a treatment for human diseases and disorders because of side effects such as tachyphylaxis. Furthermore, patients who are treated with the alkaloids develop rapid tolerance. When the body builds up a tolerance to the alkaloid, ultimately the dosage must be increased in order to get the same effects from the treatment. Unfortunately, the patient reaches a saturation point, and the treatment is harmful to the patient and does not provide any therapeutic benefit.

Currently, there is a need for low-cost, reproducible methods to isolate alkaloids and amino acids from a readily available source, as well as compositions and methods to treat or prevent a disease using the purified amino acids and alkaloids. The present invention satisfies that need. Described herein are methods for the removal of non-essential binding molecules from alkaloids and amino acids, which allow the alkaloids to be in their purest active state and thus highly effective in the treatment of disease.

### SUMMARY

The present invention provides low-cost, reproducible methods to isolate alkaloids and amino acids from a readily available source, as well as compositions and methods to treat or prevent a disease using the purified amino acids and alkaloids.

In one embodiment, a method for purification of an alkaloid is described. The method comprises first preparing a solution comprising an alkaloid. Next, the alkaloid solution is heated to a temperature between about 145° F. to about 250° F. The solution is mixed during heating. The heated, mixed solution is cooled to room temperature and filtered, resulting in a purified alkaloid. In a further embodiment, the solution is heated for at least five minutes. The filtering comprises filtering the mixture through activated carbon or a permeable membrane.

In another embodiment, a method for purification of an amino acid is described. The method comprises first preparing a solution comprising an amino acid. Next, the amino acid solution is heated to at or below its flash point. The solution is mixed during heating. The heated, mixed solution is cooled to room temperature and filtered, resulting in a purified amino acid. In a further embodiment, the solution is heated for at least five minutes. The filtering comprises filtering the mixture through activated carbon or a permeable membrane.

In one embodiment, the alkaloid comprises nicotine, caffeine, cannabis, hemp, including hemp oils, delta-9-tetrahydrocannabionol (THC), quinine, ephedrine, homoharringtonine, galantamine, vincamine, morphine, chelerythrine, piperine, psilocin, cocaine, or theobromine.

In another embodiment, the amino acid comprises alanine, arginine, asparagine, aspartic acid, cysteine, glutamine, glutamic acid, glycine, histidine, isoleucine, lysine, phenylalanine, proline, serine, threonine, tryptophan, tyrosine, or valine.

In another embodiment, a composition comprising the purified alkaloid or amino acid is described. The composition can be, for example, formulated into a beverage, a supplement, or formulated for use in e-cigarettes.

A further embodiment describes a method of treating a disease in an individual using one or more of the purified alkaloids or amino acids. In one embodiment, the disease comprises Parkinson's Disease, Alzheimer's Disease, ADHD, ADD, vascular disorders, cancer, heart disorders, migraine headaches, cluster headaches, plaque reduction or aging. In one aspect, the one or more purified alkaloids or amino acids cross the individual's blood-brain barrier.

### DESCRIPTION

### Definitions

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

Definitions of chemical terms and general terms used throughout the specification are described in more detail herein, but unless otherwise indicated the chemical elements are identified in accordance with the Periodic Table of the Elements, CAS version, Handbook of Chemistry and Physics, 75th Ed., inside cover, and specific functional groups if not specifically described herein are described by general principles of organic chemistry, as well as specific functional moieties and reactivity, as described in Organic Chemistry, 4^{th} Edition, L. G. Wade, Jr., Prentice-Hall Inc., New Jersey, 1999.

The term “alkaloid” refers to a group of naturally occurring chemical compounds that contain mostly basic nitrogen atoms. The alkaloids that can be used in the present invention include, but are not limited to, nicotine, caffeine, cannabis, and hemp, including hemp oils, delta-9-tetrahydrocannabionol (THC), quinine, ephedrine, homoharringtonine, galantamine, vincamine, morphine, chelerythrine, piperine, psilocin, cocaine, and theobromine.

The term “amino acid” includes any L or D amino acid, derived from a natural or non-natural amino acid and any analogs that are known in the art or described herein. An amino acid may be modified, for example, by the addition of a chemical entity such as a carbohydrate group, a phosphate group, a farnesyl group, an isofarnesyl group, a fatty acid group, a linker for conjugation, functionalization, or other modification, etc.

The term “buffer” or “buffered solution” refers to a mixture of acid and base which, when present in a solution, reduces or modulates changes in pH that would otherwise occur in the solution when an acid or base is added.

As used in this disclosure, the term “comprise” and variations of the term, such as “comprising” and “comprises,” are not intended to exclude other additives, components, integers ingredients or steps.

“Isolation” or “purification” as used herein means separation of alkaloids or amino acids from other components in the alkaloid or amino acid starting material, which provides a substantially pure target compound, such as a substantially pure alkaloid. A compound or molecule which is “substantially pure” contains the compound or molecule in an amount of from about 50% to about 100%, from about 50% to about 80%, from about 70% to about 85%, from about 65% to about 95% by weight of the total compound or molecule in the material processed by the method of the invention.

The term “solution” refers to a composition comprising a solvent and a solute, and includes true solutions and suspensions. Examples of solutions include a solid, liquid or gas dissolved in a liquid and particulates or micelles suspended in a liquid.

As used herein, “nicotine” refers to nicotine alkaloids, including nicotine and nicotine-like or related pharmacologically active compounds such as nor-nicotine, lobeline and the like, as well as the free-base substance nicotine and all pharmacologically acceptable salts of nicotine, including acid addition salts. Nicotine salts include nicotine hydrogen tartrate and nicotine bitartrate, as well as nicotine hydrochloride, nicotine dihydrochloride, nicotine sulfate, nicotine citrate, nicotine zinc chloride monohydrate and nicotine salicylate, either alone or in combination.

As used herein, the term “filter” refers to any type of filter, including mechanical filters that separate on the basis of size or filters that separate components of solutions, such as, for example, a charcoal or ionic filter.

The terms “individual,” “subject” and “patient” are used interchangeably herein, and generally refer to a mammal. The term “mammal” is defined as an individual belonging to the class Mammalia and includes, without limitation, humans, domestic and farm animals, and zoo, sports, and pet animals, such as cows, horses, sheep, dogs and cats.

The term “flash point” refers to the temperature at which a particular organic compound, such as an amino acid, gives off sufficient vapor to ignite in air. The flash points of each amino acid are well known to one of skill in the art.

The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.” Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; lozenges; candies; and the like.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, PBS (phosphate-buffered saline), and 5% human serum albumin. Liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined above, use thereof in the composition of the present invention is contemplated.

A “therapeutic composition” as used herein means a substance that is intended to have a therapeutic effect such as pharmaceutical compositions, genetic materials, biologics, and other substances. Pharmaceutical compositions may be configured to function inside the body with therapeutic qualities, concentration to reduce the frequency of replenishment, and the like.

As used herein, the phrases “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective can be readily determined by ordinary medical practitioner, and may vary depending on factors known in the art, such as, e.g. the type of disease, the patient's history and age, the stage of disease, and the administration of other therapeutic agents.

The term “delivery system” refers to the formulation and delivery of the composition to a patient. Delivery systems include, but are not limited to, rapid dissolvable chewable or lozenges, liquid formulation, injection, compressed powder tablets, gelcap, transdermal gel or spray, intravenous delivery, time released liquid implants, electronic/substitute cigarettes or nasal sprays.

### Alkaloids

To date, the use of alkaloids has not been a successful treatment because they are habituating, develop rapid tolerance and result in tachyphylaxis. When the body builds up a resistance to any alkaloid, ultimately the dosage must be increased in order to get the same effects from the treatment. Unfortunately, the patient reaches a saturation point, and the treatment is harmful to the patient and does not provide any therapeutic benefit.

The process and method described herein can be used to isolate the biologically active molecules in amino acids and alkaloids from inactive or binding molecules. Binding molecules could be other alkaloids or one or more amino acids. The present invention includes methods for the removal of non-essential binding molecules from alkaloids and amino acids, which allow the alkaloids to be in their purest active state and thus highly effective in the treatment of disease.

One alkaloid, nicotine, binds to and acts on the acetylcholine receptors in the brain, causes release of chemicals such as serotonin, dopamine, norepinephrine, and beta-endorphin in an individual. Nicotine can be used to treat a variety of neurological and vascular disorders. However, use of nicotine can result in tachyphylaxis in a patient.

Epidemiological research indicates that nicotine may have a neuroprotective effect; individuals who smoked were less likely to develop Alzheimer's disease and Parkinson's disease. Additionally, intermittent nicotine treatment was shown to reduce medication-induced dyskinesias by as much as 50% in models of Parkinson's disease.

Additionally, a current delivery system for nicotine is cigarettes, which is not desirable for many reasons, including that smoking is not socially acceptable. Another delivery system is a nicotine skin patch, which delivers reliable doses of nicotine to an individual. However, nicotine patches are not effective for long-term treatment of a disease or condition, because it can cause systemic side effects and because it is difficult to administer effective doses with a patch. Yet another delivery system for nicotine under development is a gel that can be applied directly to a wound site to promote angiogenesis.

It was found that by isolating the nicotine molecule (C₁₀H₁₄N₂) from impurities such as pyridine (C₅H₅N) and N-Methyl-2-pyrrolidone (C₅H₉NO), the isolated nicotine is highly effective in treatment. In the case of nicotine, pyridine and N-Methyl-2-pyrrolidone are present, but are not responsible for nicotine's biological effect.

Cannabis (THC) can be beneficial when administered to a patient. However, previous extraction methods involving alcohol-based liquid extraction results in liquefied oil-based substances that do not mix well in water-based mixtures or bind to other molecular structures. Additionally, the alcohol-base of the tincture results in the organic molecules becoming less effective over time. Furthermore, the taste, odor, and residue that result from the process are undesirable.

### Extraction Processing of Amino Acids and Alkaloids

Described herein is a method for purifying an amino acid or alkaloid from a starting material such as a commercially available alkaloid or amino acid concentrate. The starting material containing amino acids or alkaloids can be in the form of a powder or liquid concentrate, and is commercially available. The starting material contains binders or other impurities that must be removed from the amino acids or alkaloids starting material in order to obtain purified amino acids or alkaloids to use in compositions for treatment.

The starting material is first diluted in water or other solute or buffer. While continuously mixing, the temperature of the solution is slowly raised to 145° F. or above. For purification of an alkaloid, the temperature should not exceed the flash point of the alkaloid.

The purified amino acid or alkaloid solution is then physically and/or chemically filtered, resulting in a pure alkaloid or amino acid solution. The pure alkaloid or amino acid solution can be stored in water or glycerin at room temperature, or it can be refrigerated. Alternatively, the pure alkaloid or amino acid can be suspended in propylene glycol.

The purified amino acids can be diluted with water, a solvent, or a buffer prior to use in a composition.

Vapor binding is used to combine the separated sources of the alkaloids or amino acids into the composition. Vapor binding is done by heating the extracted alkaloids at or below their flash point, and injecting the heated alkaloid solution into the amino acid base. The base formulation of amino acids is continuously mixed throughout the vapor binding process, and allowed to slowly cool after heating.

### Treatment

The purified alkaloid or amino acid solution can be used to formulate a composition. The composition can be used to treat migraines and cluster headaches, as well as other disorders such as, but not limited to, Parkinson's, Alzheimer's, ADHD, ADD, vascular disorders, cancer, heart disorders, tissue detoxification (i.e. liver, mammary tissue, spleen), plaque reduction and aging.

A severe headache, or aggregated headache such as a migraine, is a headache with intense, throbbing pain. Migraines can also cause nausea and sensitivity to light and sound in an individual. It is thought that migraine pain is triggered by swollen blood vessels within the brain case, including certain nerves inside the brain matter. Two types of migraines have been described—a classic migraine is a migraine with aura, and a common migraine is a migraine without aura. The pain phase of a classic migraine is typically preceded by the aura, which is a visual disturbance that partially or completely fills the patient's field of vision. The aura can last from five to sixty minutes, followed by a phase of intense cranial pain which can be accompanied by nausea and sensitivity to both light and sound, which can last several hours.

More than 28 million people in the United States suffer from migraines. Migraines occur most often in adults age 25 through 55 years old. Women are three times more likely than men to have a migraine.

Current treatment of migraine and cluster headaches include triptans such as sumatriptan, rizatriptan, naratriptan, zolmitriptan, eletriptan, almotriptan, trovatriptan, frovatriptan, and avitriptan, which are serotonin (or 5HT) agonists. Triptans work in part to constrict the blood vessels in the brain, thus relieving swelling of the brain tissues and treating symptoms of migraines. However, while triptans are effective to treat headaches, triptans can neither prevent nor cure migraine or cluster headaches.

Other treatments have been described, including natural or alternative treatments such as magnesium, ginger, ginkgo biloba, feverfew, and melatonin (see e.g. U.S. Pat. No. 6,068,999) or tobacco (see e.g. U.S. Pat. No. 7,070,817). However, these treatments have not always led to consistent or significant results in patients.

Administration of the amino acid tryptophan in the form of 5-Hydroxytryptophan (5-HTP) has shown only moderate effects in migraine prevention, and has been shown to be ineffective in aborting or significantly mitigating pain after onset of a migraine. 5-HTP is thought to be beneficial to minimize the frequency, intensity and duration of migraines if used daily as a preventative (see e.g. U.S. Pat. No. 5,939,076). The inability of 5-HTP to abort or mitigate migraine pain seems to lie in the peripheral metabolism of orally delivered 5-HTP by the enteric nervous system.

There remains a need for an effective treatment to prevent or reduce the triggers that cause migraine headaches and cluster headaches. Described herein is a composition that increases the production of dopamine and serotonin, as well as vascular constrictor that is delivered to the brain and central nervous system, and can be used to treat headaches, including migraine and aggregated headaches during outset.

The composition of the invention can be administered orally after the onset of migraine symptoms. For example, the composition can be taken as two or three units of the mixture over a period of 20 minutes after the onset of migraine symptoms.

The composition can prevent the onset of headache or migraine symptoms when administered on a regular, consistent basis.

### Electronic Cigarettes

The electronic cigarette (e-cigarette), or vaping, industry is a multi-billion dollar industry in the United States. E-cigarettes typically contain 1 mg to 3 mg of nicotine per unit. Glycerin is also added to keep the nicotine from dehydrating, and keeping the nicotine in solution. Other flavors such as peach and cherry, as well as coloring can also be added.

While e-cigarettes are regulated by the FDA in the United States, it is generally believed that e-cigarettes are less harmful than regular cigarettes, but still provide the same biological effect as regular cigarettes.

The present invention uses purified alkaloids and amino acids to provide a composition containing one or more alkaloids or amino acids, as well as additives such as flavorings and vitamins to be used in e-cigarettes. In one aspect of the invention, the composition for use in e-cigarettes contains nicotine, and a liquid vaping base such as, for example, water or glycerin. In addition, the composition can contain additional alkaloids and amino acids. It is contemplated that one formulation of the composition is timed-release or slow-release in order to give the user a controlled energy lift.

It was found that using between 6 and 15 puffs, or vapes, of the composition gave the users an energy lift without the side effects associated with canned energy drinks.

### EXAMPLES

### Example 1

### Purification of Nicotine

Concentrated nicotine is commercially available. One manufacturer is Spectrum Chemical Manufacturing Corp. (New Brunswick, N.J.), which sells liquid nicotine concentrate as 1000 mg of nicotine per milliliter (mL). However, the commercially available nicotine contains impurities such as pyridine and N-Methyl-2-pyrrolidone, and as such the nicotine must be extracted from the pyridine and N-Methyl-2-pyrrolidone. One such extraction method is set forth below.

In this example, a nicotine solution was made by a ten-fold dilution of 1000 mg/ml nicotine concentrate (Spectrum Chemical Manufacturing Corp.) with a solvent such as water. The nicotine solution was continuously mixed, while the temperature of the nicotine solution was rapidly increased to 185° Ft. Once the nicotine solution reached 185° F., the heat was turned off, and the heated nicotine solution was cooled to room temperature with continuous mixing.

The nicotine solution is then filtered through a submicron filter, resulting in a pure nicotine solution. The pure nicotine solution can be stored in water or glycerin at room temperature or refrigerated.

Clinical studies show that after extraction, nicotine can be used for treatment without side effects such as tachyphylaxis.

### Example 2

### Purification of Tryptophan

Amino acids are commercially available. One manufacturer is Spectrum Chemical Manufacturing Corp. (New Brunswick, N.J.). However, the commercially available amino acids contain unwanted binders which must first be removed prior to use as set forth below in a process referred to as the vapor binding process.

First, the commercially available amino acid, such as tryptophan, is mixed with a solvent such as water. During mixing, the amino acid solution is heated to 145° F. or higher. Once the amino acid solution reaches the target temperature, the heat is removed and the amino acid solution is cooled to room temperature with continuous mixing.

The extracted, vaporized amino acid solution is optionally filtered using a micron filter during the cooling period.

The purified amino acids can be diluted with water, glycerin, a solvent, or a buffer prior to use.

### Example 3

### Composition

After the nicotine is purified using the extraction process, it can be combined and/or attached to additional molecules such as, for example, amino acids and alkaloids. The purified nicotine can be used to deliver the attached molecules to the brain for treatment, for example, for treatment of migraine headaches.

In one formulation, a 500 mg nicotine lozenge was prepared. The starting material was the nicotine prepared as the pure nicotine solution described above, which was added to 100 mg tryptophan, 150 mg caffeine, 75 mg tyrosine, 50 mg phenylalanine and 5 mg of lithium were homogenized at 33,000 rpm.

### Example 4

### Purification of THC from Cannabis

Before purification, hash will be extracted from cannabis using commonly used methods, such as “honey bee extraction” which results in a sticky extract that is high in THC, CBD and other cannabinoids.

A solute such as water is added to the extracted hash, and the solution mixed at high speed while simultaneously heating the mixture to at least 392° F. The mixture is then allowed to cool and filtered with a sub-micron filter.

After filtration, the solution contains THC and trace elements of CBD and other cannabinoids.

Although the present invention has been described in considerable detail with reference to certain preferred embodiments, other embodiments are possible. The steps disclosed for the present methods, for example, are not intended to be limiting nor are they intended to indicate that each step is necessarily essential to the method, but instead are exemplary steps only. Therefore, the scope of the appended claims should not be limited to the description of preferred embodiments contained in this disclosure.

What is claimed is:

1. A method for purification of alkaloid, the method comprising:
a) preparing a solution comprising one or more than one alkaloid;
b) heating the alkaloid solution to a temperature between about 145° F. to about 250° F.;
c) mixing the alkaloid solution during heating;
d) cooling the mixed alkaloid solution; and
e) filtering the cooled alkaloid mixture;
thereby purifying the alkaloid.

2. The method of claim 1, wherein the alkaloid comprises nicotine, caffeine, cannabis, hemp, including hemp oils, delta-9-tetrahydrocannabionol (THC), quinine, ephedrine, homoharringtonine, galantamine, vincamine, morphine, chelerythrine, piperine, psilocin, cocaine, or theobromine.

3. The method of claim 1, wherein the alkaloid solution is heated for at least five minutes.

4. The method of claim 1, wherein the filtering comprises filtering the mixture through activated carbon.

5. The method of claim 1, wherein the filtering comprises filtering the mixture through a permeable membrane.

6. A composition comprising the purified alkaloid of claim 1.

7. The composition of claim 6, wherein the composition comprises a beverage.

8. The composition of claim 6, wherein the composition comprises a supplement.

9. The composition of claim 6, wherein the composition is formulated for use in e-cigarettes.

10. A method of treating a disease in an individual comprising administering a therapeutically effective amount of the composition of claim 6 to an individual in need of such treatment.

11. The method of claim 10, wherein the disease comprises Parkinson's Disease, Alzheimer's Disease, ADHD, ADD, vascular disorders, cancer, heart disorders, migraine headaches, cluster headaches, plaque reduction or aging.

12. A method for purification of an amino acid, the method comprising:
a) preparing a solution comprising one or more than one amino acid;
b) heating the amino acid solution to a temperature at or below the flash point of the amino acid;
c) mixing the amino acid solution during heating;
d) cooling the mixed amino acid solution; and
e) filtering or homogenizing the cooled amino acid mixture;
thereby purifying the amino acid.

13. The method of claim 12, wherein the amino acid comprises alanine, arginine, asparagine, aspartic acid, cysteine, glutamine, glutamic acid, glycine, histidine, isoleucine, lysine, phenylalanine, proline, serine, threonine, tryptophan, tyrosine, or valine.

14. The method of claim 12, wherein the amino acid solution is heated for at least five minutes.

15. The method of claim 12, wherein the filtering comprises filtering the mixture through activated carbon.

16. The method of claim 12, wherein the filtering comprises filtering the mixture through a permeable membrane.

17. A composition comprising the purified amino acid of claim 12.

18. The composition of claim 17, wherein the composition comprises a beverage.

19. The composition of claim 17, wherein the composition comprises a supplement.

20. The composition of claim 17, wherein the composition is formulated for use in e-cigarettes.

21. A method of treating a disease in an individual comprising administering a therapeutically effective amount of the composition of claim 17 to an individual in need of such treatment.

22. The method of claim 21, wherein the disease comprises Parkinson's Disease, Alzheimer's Disease, ADHD, ADD, vascular disorders, cancer, heart disorders, migraine headaches, cluster headaches, plaque reduction or aging.
